(19) United States
(12) Patent Application Publication
(10) Pub. No.: US 2012/0265138 A1
(43) Pub. Date: Oct. 18, 2012

(54) Apparatus for use in injecting insulin from a filled syringe

(76) Inventors: Stanley Harylka, Holmdel, NJ (US); Margaret A. Herchakowski, Shrewsbury, NJ (US)

(21) Appl. No.: 13/066,522

(22) Filed: Apr. 18, 2011

(51) Int. Cl. A61M 5/42

(52) U.S. Cl. 604/116

(57) ABSTRACT

A Guide for injecting insulin in the abdomen, thigh or arm according to dimension including a wrap-around band and a series of spaced apertures along its length, with the openings of the apertures being sized to receive an insulin dispensing pin of a filled syringe, and with a reminder indicating at which site an injection was last made so as to reduce the possibility of scar tissue or abscess development for senior citizens, especially, forgetting where the most recent injection was made.

### CROSS-REFERENCE TO RELATED APPLICATIONS

NONE

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

NOT APPLICABLE

### BACKGROUND OF THE INVENTION

1. Field of the Invention

This invention relates to the medical treatments of diabetics, in general, and to apparatus for injecting insulin from a filled syringe into the abdomen, thighs or arms of a Type 1 diabetic, in particular.

2. Description of the Related Art As is well known and understood, diabetes is a chronic, lifelong disease marked by high levels of sugar in the blood. As is also known, the most common types of diabetes are classified as Type 1 and Type 2, with Type 2 diabetes being far more prevalent. With Type 2, the pancreas does not make enough insulin to keep blood glucose levels normal, often because the body does not respond well to insulin. Symptoms of Type 2 diabetes include blurred vision, fatigue, increased appetite, increased thirst and increased urination—and are treated with exercise, diet, and medications taken orally.

People with Type 1 diabetes, on the other hand, are unable to make their own insulin, and thus need daily insulin injections as insulin does not come in pill form. Because injections are generally needed one to four times per day, many Type 1 diabetics use an insulin pump, worn at all times to deliver a steady flow of insulin throughout the day; others may use inhaled insulin as an alternative. As insulin preparations differ in how soon they begin to work and how long they last, the Type 1 diabetic's health care professional often instructs the users as to how to give the injections themselves, under the skin. While injections of this nature can be given in the thigh or arm areas, faster action normally follows injections in the abdomen. By age 14, it has been accepted, most Type 1 diabetics are able to give their own injections.

Experience has shown, however, that where repeat injections are needed on a daily basis, senior citizens oftentimes forget the location on the body where they last gave the insulin injection. Analysis has shown that repeated injections at the same general local can give rise to the formation there of scar tissue or abscesses, as well as to lead to possible infection. For such reason a Guide would be helpful to assist in injecting insulin into the abdomen, thigh or arm so as to reduce the possibilities of these undesired occurrences—and, as an aid to senior citizens in particular, whose remembrances and recollections wane the most.

And, this is so, whether the diabetic insulin user fills the injection syringe himself/herself from a bottle of insulin, or whether he/she uses an obtained prefilled syringe containing multiple doses, now becoming available.

### OBJECTS OF THE INVENTION

It is an object of the present invention, therefore, to provide a new and improved apparatus for injecting insulin from a filled syringe for Type 1 diabetic treatment.

It is an object of the present invention, also, to provide such apparatus for injecting the insulin into the abdomen, thigh or arm of the diabetic.

It is another object of the invention, to provide such apparatus no matter how often the diabetic requires the insulin injections during the course of a day.

And, it is a primary object of the invention to provide this apparatus in a manner so as to minimize the possibility of scar tissue, abscess or infection that might otherwise form or result from repeated administrations of the insulin medication at the same localized site.

### SUMMARY OF THE INVENTION

As will become clear from the following description, the apparatus of the invention proceeds from a recognition that each injection site of the syringe needs some degree of time to heal before injecting again in that same site. Thus, and as will be understood from the writings below, the apparatus of the invention includes a wrap-around band with a series of spaced apertures along its length, each of a size to receive an insulin dispensing pin of the syringe. For ease of putting on, the wrap-around band could be of an adjustable length to accommodate the abdomen, thigh or arm of the diabetic, or could be of a fixed size dimensioned to meet that portion of the body, and of an elastic fabrication. The series of spaced apertures are, at the same time, formed by a setting of grommets, preferably of plastic so as to allow the band to be easily washed and laundered. (Metal grommets, on the other hand, have a tendency to rust.)

In a preferred embodiment for abdominal injections, some 14 left-to-right spaced grommet apertures are provided, with a demarkation indicator dividing the series into left and right half-portions. (In such manner, the demarkation indicator would be marked for placement adjacent the navel of the wearer in dividing the abdomen into left and right sections. In accordance with this preferred embodiment, the grommets are selected of substantially ⅜ inch diameter, with the spacing of the grommets being substantially 1 inch apart. The wrap-around band then can serve as a guide for injecting the insulin—and while especially attractive for injecting the insulin into the abdomen, can alternatively be sized for injections in the thigh or arm areas simply by choice of the length of the band.)

### BRIEF DESCRIPTION OF THE DRAWINGS

These and other features of the present invention will be more clearly understood from a consideration of the following description, taken in connection with the accompanying drawings, in which:

FIG. 1 is a depiction of a prefilled syringe available for the insulin injection of Type 1 diabetics; and

FIG. 2 is an illustration of the wrap-around band with its spaced apertures operable with the syringe of FIG. 1.

### DETAILED DESCRIPTION OF THE INVENTION

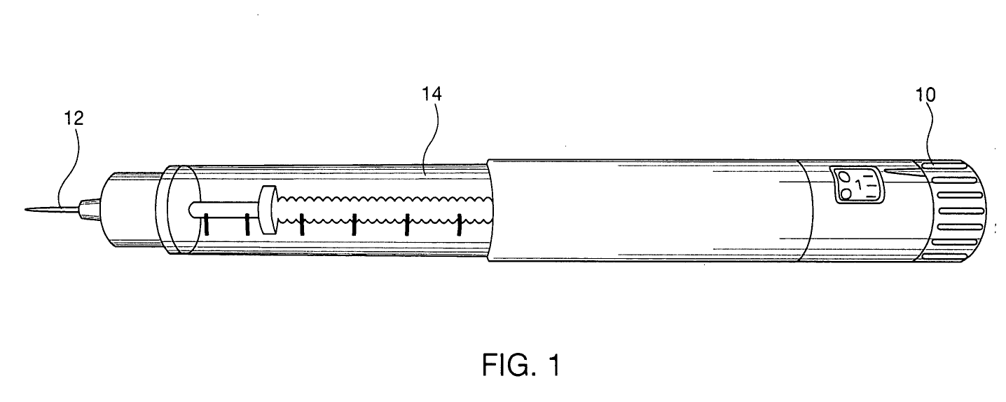

FIG. 1 illustrates an insulin injecting prefilled syringe for Type 1 diabetics manufactured by Novo Nordisk of DK-2880, Bagsvaerd, Denmark. The syringe may be of a type identified as NovoLog Mix 70/30 FlexPen, containing 70% insulin as part protamine suspension and 30% insulin as part injection (although other prefilled, or individual user filled insulin syringes will work with the present invention). With the NovoLog model shown, a knurled knob 10 is adjustable in selecting the number of units of insulin to be injectable in any given application. Its insulin dispensing pin is shown at 12, and the syringe itself 14 is typically packaged 5 syringes to a box. With a dialed-in selection of 8 insulin units per injection, some 10 individual injections could be had before emptying the syringe.

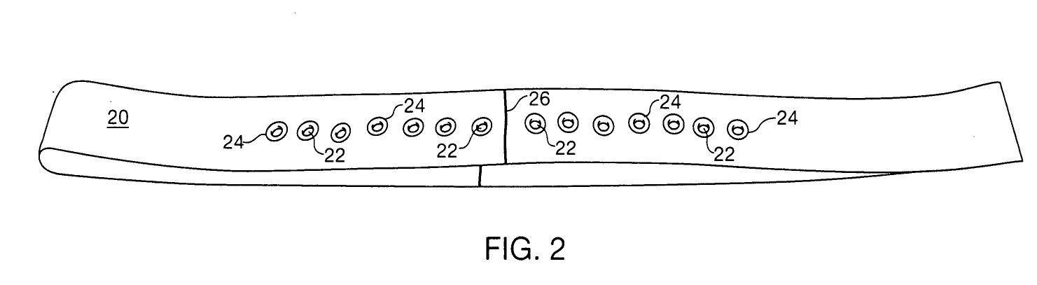

The wrap-around band of the invention is shown at 20 in FIG. 2—and, as previously indicated, may be sized to wrap-around the abdomen, thigh or arm of the diabetic user. To accommodate, and/or deal with the forgetfulness or other impairment of a senior-aged Type 1 diabetic, a series of spaced apertures 22 are shown along the band's length, running and extending from left-to-right. With the opening of the apertures sized to receive the tip of the dispensing pin 12 of the syringe, a user administered injection of insulin of prescribed dosage can easily follow.

As will be appreciated, however, repeated injections at the same site location can well lead to scar tissue and abscess formation, and possible infection; thus the spaced apertures allow the user to select an individual aperture for a first injection, its adjacent aperture for a second injection, the aperture adjacent to that for a third injection, and so forth. Starting either from the left side proceeding to the right, or from the right proceeding to the left, the only decision a user needs to make is to remember that the procession is left-to-right, or right-to-left—and to somehow mark the aperture in which the injection was last made. This can be as simple as covering over each last aperture used as each administration is completed.

In accordance with the invention, the apertures are formed by a setting of grommets 24 of preferably plastic fabrication to permit the wrap-around band with the grommets to be periodically washed or otherwise laundered. Here, the grommets are selected of a ⅜ inch diameter, with them being individually spaced 1 inch from each another. When used as a band to wrap around a user for abdominal injections, a demarkation indicator 26 is provided to align with the navel in identifying left-and-right halves of the band. In proceeding from side-to-side in the embodiment of FIG. 2, a diabetic insulin user then has 14 different grommet apertures available for injection. Such would suffice for a user injecting himself/herself with insulin one time per day, for a two week measure of injections. Further, by making the wrap-around band construction substantially 2 inches in width, the band can additionally be slid upwardly or downwardly with respect to the initial abdomen positioning so as to essentially establish a second line of possible injection locations above the first, and a third line below the first to easily set up 42 individual locations where injections could be made. In such arrangement, the 7th placed aperture 22 from the left and the 7th placed aperture from the right could be spaced a distance apart greater than 1 inch in a final manufacture. And, as will be appreciated by those skilled in the art, since diabetes is a chronic and lifelong disease that affects a high percentage of any population, the apparatus of the invention will be seen to be usable with great effectiveness both by old and young alike.

To facilitate sliding the band upwardly or downwardly along the abdomen, and to fit users of different body dimension, the wrap around band 20 is of a stretchable construction, (for example, of a 60 inch length) preferably of an elastic fabrication. It can be manufactured with an adjustable clasp or Velcro fastening for purposes of sizing, or of an extra-large, large, medium or small fixed size—for use on an abdomen, thigh or arm body portion where desired.

While there has been described what is considered to be a preferred embodiment of the present invention for prefilled or user filled insulin injection syringes, it will be readily understood that modifications can be made without departing from the scope of the teachings herein. For at least such reason, therefore, resort should be had to the claims appended hereto for a true understanding of the invention.

1. Apparatus for use in injecting insulin to the abdomen from a dispensing pin of a filled syringe comprising:
a band; and
a series of spaced apertures along a length thereof;
with the band being of a length to wrap around the abdomen of a diabetic user, with the apertures being of a size to receive the dispensing pin of the filled syringe;
with the band including 14 spaced apart apertures extending along its length left-to-right, within an outwardly facing front surface of the band including a band placement demarkation indicator dividing the 14 left-to-right apertures substantially half-ways in length, and with the demarkation indicator being visible for placement in alignment with the navel of the diabetic user for the injection of insulin in either the left-or-right side of the user's abdomen on a preselected time basis.

2. The apparatus of claim 1 wherein said band is composed of a stretchable construction.

3. The apparatus of claim 2 wherein said band is composed of an elastic fabric.

4. The apparatus of claim 1 wherein said apertures are composed of a series of grommets.

5. The apparatus of claim 4 wherein said apertures are composed of a series of plastic grommets.

6. the apparatus of claim 1 wherein said band is comprised of an elastic fabric and wherein said apertures are composed of a series of plastic grommets.

7. The apparatus of claim 6 wherein said wrap-around band includes 14 spaced apart apertures extending left-to-right.

8 The apparatus of claim 7 wherein said wrap-around band includes a demarkation indicator dividing said 14 left-to-right apertures half ways in length.

9. The apparatus of claim 8 wherein said band is of a substantially 2 inch width.

10. The apparatus of claim 9 wherein said grommets are of a substantially ⅜ inch diameter.

11. The apparatus of claim 9 wherein individual ones of each of said left half and right half grommets are spaced substantially 1 inches apart.

12. The apparatus of claim 9 wherein said band is adjustable downward in length from an initial 60 inch length.

13. Apparatus for use in injecting insulin to the abdomen from a dispensing pin of a filled syringe comprising:
a band; and
a series of spaced apertures along a length thereof;
with this band being of a length to wrap around the abdomen of a diabetic user, with the apertures being of a size to receive the dispensing pin of the filled syringe;
with the band including 14 spaced apart apertures extending along its length left-to-right, with an outwardly facing front surface of the band including a band placement demarkation indicator dividing the 14 left-to-right apertures substantially half-ways in length, and with the demarkation indicator being visible for placement in alignment with the navel of the diabetic user for the injection of insulin in either the left-or-right side of the user's abdomen on a preselected time basis;
wherein the band is of an adjustable length, wherein the band is of a substantially 2 inch width, wherein the apertures along the length of the bans are composed of a series of grommets, and wherein individual ones of the grommets on each of the left and right halves of the band placement demarkation indicator are spaded substantially 1 inch apart.

14. The apparatus of claim 13 wherein the band is composed of a stretchable, washable elastic fabric.

15. The apparatus of claim 14 wherein the band is adjustable to a length of 60 inches to accommodate the abdomen size of most diabetic users thereof.
